(19) United States
(12) Patent Application Publication
(10) Pub. No.: US 2023/0072582 A1
(43) Pub. Date: Mar. 9, 2023

(54) METHOD AND SYSTEM FOR SUPPORTING AN MRT EXAMINATION

(71) Applicant: Siemens Healthcare GmbH, Erlangen (DE)

(72) Inventors: Stephan NUFER, Erlangen (DE); Julian WOHLERS, Erlangen (DE); Thomas KUNZ, Fuerth (DE)

(73) Assignee: Siemens Healthcare GmbH, Erlangen (DE)

(21) Appl. No.: 17/899,739

(22) Filed: Aug. 31, 2022

(30) Foreign Application Priority Data
Sep. 3, 2021 (DE) 10 2021 209 750.2

(51) Int. Cl. A61B 5/055; A61B 5/00

(52) U.S. Cl. CPC A61B 5/055; A61B 5/742; A61B 5/746; A61B 5/7475

(57) ABSTRACT

One or more example embodiments relates to a method for supporting an MRT examination of a patient via a coil placed on the patient. One or more example embodiments also relates to apparatus, a control facility for an MRT system and also an MRT system.

### CROSS-REFERENCE TO RELATED APPLICATION(S)

The present application claims priority under 35 U.S.C. § 119 to German Patent Application No. 102021209750.2, filed Sep. 3, 2021, the entire contents of which are incorporated herein by reference.

### FIELD

One or more example embodiments of the present invention relates to a method and a system for supporting an examination within the framework of magnetic resonance tomography (MRT) of a patient via a coil placed on the patient. Preferably this method serves to control an MRT system and in particular allows visual feedback of the coil positioning and orientation via a sensor system during MRT examinations.

### BACKGROUND

Many MRT examinations are carried out nowadays with an, in particular flexible, coil not fixed in one position, which is placed on a patient. In this case the coil is positioned on the patient so that the center of the anatomy to be examined (of the examination region) corresponds to the center of the coil. Under these circumstances a flexible coil offers the advantage of being able to be used for examination of different parts of the body. If the coil is connected to the MRT system, often a number of anatomies are displayed or provided for the isocentering at the operator interface.

A user has to actively report the corresponding anatomy that they wish to examine to the system, so that the latter knows how far the table must be moved into the isocenter. The position must first be chosen or selected for this before the table movement into the isocenter is started. If an MRT system is now equipped with a table that has a restricted range of movement (for examining a part of the body), then it can occur that the coil is moved in the Z direction (longitudinal axis) and comes to rest in a region that, because of the limited table movement, cannot be moved into the isocenter and thus no imaging is possible.

The size of the patient further also plays a role in whether or not an anatomy can be moved in a specific orientation (feet or head first) into the isocenter. The user might only first notice this when entering the isocenter manually at the scanner operator interface or during the patient registration at the console (main operator interface). The result of this is that the patient must be positioned or prepared again, which means using up valuable time in a tightly scheduled radiological workflow and naturally is also uncomfortable for the patient themself. This can also lead to problems when the patient has been sedated beforehand or has already received contrast media before the measurement, since here corresponding time intervals must be adhered to between administration of the medium and start of the measurement.

Moreover it is not certain that the coil is also correctly positioned on the anatomy to be examined. With inexperienced users in particular it can occur that the coil is either offset in the Z axis (offset between center of the coil and center of the anatomy to be examined), the wrong side of the patient is covered (right instead of left) or a rotation about the Y axis is present. The former can result in the desired anatomy coverage not being achieved and following steps in the image processing not being successful (for example auto align). The latter can lead to SNR (signal-to-noise ratio) problems or reduction when using acceleration technologies for the MRT imaging.

As regards the position of the coil or the center of the coil, nowadays this can also be defined using laser sight or a line on the table. The latter absolutely requires the manual entry of the value read off from the line at the operator interface.

### SUMMARY

Both methods thus represent an additional operating step and an additional source of errors.

One or more example embodiments of the present invention specifies an alternative, more convenient method or system for supporting an MRT examination and in particular for controlling a magnetic resonance tomography system for creation of image data, with which the disadvantages described above are avoided.

According to one or more example embodiments, a method for supporting an MRT examination of a patient via a coil placed on the patient includes providing of a protocol for the MRT examination; providing patient information, the patient information comprising at least information about body dimensions and orientation of the patient; measuring a location of the coil via an automated data acquisition by a measurement facility; automatically determining an examination region of the patient from the protocol and the patient information provided and a required location of the coil; automatically determining the location of the coil relative to the required location based on the location of the coil measured; comparing the relative location of the coil determined with a predetermined boundary region around a predetermined required location of the coil; and outputting output data based on the relative location of the coil when the relative location of the coil lies outside the boundary region.

According to one or more example embodiments, the coil is a flexible coil.

According to one or more example embodiments, the measuring the location of the coil includes determining an isocenter of the coil from the measured location of the coil, a lateral distance from the isocenter to the examination region being the location of the coil, and the outputting includes outputting the lateral distance when the lateral distance is greater than a limit value.

According to one or more example embodiments, the method further includes determining a twisting of the coil in one plane or in space with regard to a predetermined coordinate system based on the measured location of the coil; and comparing the determined twisting with a predetermined maximum twisting in the predetermined coordinate system, wherein the outputting outputs the output data when the determining twisting is greater than the maximum twisting.

According to one or more example embodiments, the measurement facility comprises at least one measurement element, the measurement element being a Hall effect sensor, a Tilt sensor, a camera, an ultrasound sensor, a radar sensor or a near field sensor, the at least one measurement element is arranged on at least one of the coil or a camera.

According to one or more example embodiments, the output data includes the location of the coil determined.

According to one or more example embodiments, the method further includes providing a visualization of the coil based on the output data.

According to one or more example embodiments, the outputting outputs at least one of a visual warning, an acoustic warning or information when the relative location of the coil lies outside the boundary region or a measurement region of the MRT examination, at least one of a visual warning, an acoustic warning or information indicating how the coil is to be positioned such that the relative location of the coil lies within at least one of the boundary region or a measurement region.

According to one or more example embodiments, the output data is used to adjust the protocol to the location of the coil.

According to one or more example embodiments, an MRT examination only takes place when the relative location of the coil lies within the boundary region or there is a manual input by an operator.

According to one or more example embodiments, an apparatus for supporting an MRT examination of a patient via a coil placed on the patient includes a first data interface configured to receive a protocol for the MRT examination and patient information, the patient information comprising at least information about body dimensions and orientation of the patient; a measurement facility configured to measure a location of the coil via automated data acquisition; a determination unit configured to automatically determine an examination region of the patient from the protocol determined and the patient information, a required location of the coil above the examination region, and the location of the coil relative to the required location based on the location L of the coil determined; a comparison unit configured to compare the relative location of the coil determined with a predetermined boundary region around a predetermined required location of the coil; and a second data interface configured to output of output data based on the relative location of the coil when the location of the coil lies outside the boundary region.

According to one or more example embodiments, a control facility is configured to control a magnetic resonance tomography system, which is configured to carry out a method of one or more example embodiments.

According to one or more example embodiments, a magnetic resonance tomography system comprising the control facility.

At least one example embodiment provides a non-transitory computer program product with a computer program with program sections for carrying out a method of one or more example embodiments when the computer program is executed in a computing system or a control facility of a magnetic resonance tomography system.

At least one example embodiment provides a non-transitory computer-readable medium including program sections that, when executed by a computer unit, cause the computer unit perform a method of one or more example embodiments.

### BRIEF DESCRIPTION OF THE DRAWINGS

The invention will be explained once again in greater detail below with reference to the enclosed figures with the aid of exemplary embodiments. In this case the same components are labeled with identical reference numbers in the different figures. As a rule the figures are not true-to-scale. In the figures:

FIG. 1 shows a schematic diagram of a magnetic resonance tomography system in accordance with an exemplary embodiment of the invention,

FIG. 2 shows a flow diagram for a possible execution sequence of an inventive method in accordance with an exemplary embodiment of the invention,

FIG. 3 shows a patient with a flexible coil on a couch in accordance with an exemplary embodiment of the invention,

FIG. 4 shows an example for a display of a patient, a coil, and an examination region in accordance with an exemplary embodiment of the invention,

FIG. 5 shows an example for an incorrectly positioned coil,

FIG. 6 shows an example for a correctly positioned coil in accordance with an exemplary embodiment of the invention, and

FIG. 7 shows in an example for a twisted coil in accordance with an exemplary embodiment of the invention.

Only the elements of significance for the invention or helpful in understanding it are depicted in the following figures. Thus for example no slice selection gradients are shown, although they can certainly be present in the pulse sequence.

### DETAILED DESCRIPTION

One or more example embodiments is used for supporting an MRT examination of a patient via a coil placed on the patient (i.e. not fixed in one position) and in particular for controlling an MRT system. It comprises the following steps:
provision of a protocol for the MRT examination, provision of (in particular comprehensive) patient information, at least information about body measurements and orientation (feet first vs. head first) and in particular also weight of the patient,
measurement of a location of the coil via automated data acquisition (location measurement) via a measurement facility,
automatic determination of an examination region of the patient from the protocol provided and the patient information and a required location of the coil above the examination region,
automatic determination of the location of the coil relative to the required location based on the location of the coil determined,
comparison of the relative location of the coil determined with a predetermined boundary region around a predefined required location of the coil,
output of output data, based on the relative location of the coil at least in the case that the location of the coil lies outside the boundary region.

Protocols for the MRT examination are the prior art and generally known. They specify the pulse sequences to be used for an MRT system and generally define the examination region as well as the sequence of the examination.

Patient information is likewise known. It relates to data about the patient, for example size, weight, age, gender. For the method it is necessary to obtain at least information about body dimensions of the patient and orientation of the patient (on the table / the patient support). In the simplest case this can be a body size or the notification that a predetermined patient is to be examined (wherein then the corresponding data for the patient must be known). More precise data, for example the proportions of the body or the precise location of organs to be examined, is especially preferred.

This data must be available for the steps that follow and can be entered manually or retrieved automatically. If a protocol has been selected automatically or selected by a user for example for an MRT examination and a dataset is available about the patient to be examined, the method should be furnished with this information or should be able to retrieve it.

The term “location” of the coil means its location on a plane (in particular the X-Z plane of an MRT scanner) or in space. In this case the term location relates to the lateral position of the coil and/or the twisting of the coil, in particular to both aspects. Thus “location” means position and/or orientation of the coil. It should be pointed out here that Z lies in the longitudinal axis of the scanner, X in the horizontal and Y in the vertical, each orthogonal to the longitudinal axis. For a patient lying on their back in the scanner the X-Z plane would correspond to the frontal plane of the patient.

For the measurement of the location this should take place with regard to a defined coordinate system for the MRT system, in particular relative to the patient table, since the patient is lying there and the examination region is thus also located there. The measurement takes place via automated data acquisition, i.e. by a measurement that automatically creates digital measurement data, which can be processed directly by a computing system. The result of this is that the location is available directly to the method without a manual entry. A camera or Hall effect probe (or Hall effect sensor) attached to a coil or also a tilt probe (or sensor) can serve as a measurement facility in this case. The location can be produced directly from measured values or can be computed from measured values.

The examination region of the patient (by which the region of the patient to be examined, i.e. for example an organ or region of the body, is meant) is determined from the protocol and the patient information provided. The protocol in this case specifies the abstract examination region (for example “left shoulder”, “head” or “liver”). The patient information specifies the exact position of the examination region for the actual examination. This determination should also take place relative to the aforementioned coordinate system (for example to the MRT scanner or to the patient table), so that a comparison with the coil location becomes easier later. It is preferred that the position of the examination region is determined in the same coordinate system as the location of the coil.

As a rule the head position of a patient in the MRT scanner or on the patient table is clearly defined. If the protocol now specifies that a liver examination is to be carried out, the patient information indicates where the liver for the current patient should be localized relative to the MRT scanner or to the patient table. This position in this case would be the examination region of the patient.

The required location of the coil above the examination region is given by the position of the examination region determined and the known dimensions of the coil and MRT scanner. As regards a lateral position, the isocenter of the coil should match the examination region, or at best lie precisely in the center of it. As regards a twisting of the coil, this should lie so that the signal-to-noise ratio is optimal, or the k-space can be filled optimally with measured values. A rectangular coil lies in a preferred case so that its sides are aligned orthogonally or parallel to the longitudinal axis of the MRT scanner (Z axis).

Since the optimal location of a coil for different examinations is known, the required location is in principle produced automatically from the examination region of the patient determined. It can be specified for example that the isocenter of the required region should lie in the center of the examination region of the patient. It can however also be specified that a number of coils are used, of which the required location is known from a predetermined arrangement, for example when a large region or a plurality of smaller regions is to be covered. The required location then relates in particular to the desired locations of the individual coils. The method can be run a number of times for a plurality of coils for an individual coil in each case (the required location is then the location of the respective coil and the boundary region relates to this coil) or can be run a single time, wherein all coils are taken into account (required location and boundary region relate to all coils). A combination is also possible, in which the method is run a number of times for a part of the coils in each case (required location and boundary region relate to all coils considered). Whether one or more coils are used could be determined on the basis of the weight of the patient (where necessary in relation to body size). It is therefore preferred that the weight is part of the patient information.

In the determination of the location of the coil relative to the required location, the lateral distance of the location to a required position, for example a required isocenter, is determined and/or a twisting of the coil with respect to a required orientation is determined. In principle, what is determined is the extent to which the real location differs from the required location. This always takes place based on the location of the coil determined. In principle, the relative location is a (vectoral or scalar or more complex) difference between location and required location.

This relative location of the coil is now compared with a predetermined boundary region around the predetermined required location of the coil. In this case just a distance can be compared with a predetermined limit value and/or an angle of twist with a predetermined limit angle. Limit value and/or limit angle in this simple case would determine the boundary region. The boundary region can however also be a more complex object, for example an abstract space, a point amount or a vector amount. It is important that the boundary region predetermines a region for a still tolerable location of the coil. It should be pointed out that in practice twisting angle or vector amounts do not absolutely have to be displayed to the user, but preferably a visual example for the correct scenario is offered, for example a graphical representation of a correct position. Thus in particular a learning effect can be achieved.

It should likewise be pointed out here that the required location or the boundary region is not absolutely restricted to the examination region (even if this should always be taken into account), but also to the measurement region of the MRT system. It is preferred that consideration is also given to whether the examination region or the location of the coil always lies within the measurement region. In this case the required location or the boundary region should always lie within the measurement region, so that when the coil is positioned outside the measurement region a warning can be output. The output data could be different in this case than for a deviation from the examination region, for example the message “coil outside the measurement region!”.

In principle, as regards the possible location of the coil, there are three different scenarios:
1. The coil can carry out the measurements defined by the protocol in the examination region and the desired MRT images can be made,
2. The coil can only inadequately carry out the measurements defined by the protocol and no satisfactory MRT images can be made, yet the coil is still located on the table or in the measurement region of the scanner, however (for the untrained user) it is not immediately evident that no images can be made,
3. No MRT images or only inadequate images can be made, the coil lies outside the measurement region (for example for an extreme position of the coil at the edge).

It is preferred that an additional check is made, with a required location or such a boundary region taking account of the measurement region, as to whether a desired examination region (an anatomy) lies in the measurement region of the MRT scanner at all. When a coil is placed above a region that cannot be moved at all into the isocenter or only into a region of the scanner with inadequate magnetic field homogeneity, the user can be alerted at an early stage or it might if necessary be suggested that another orientation (for example feet-first instead of head-first) is tried.

The output data is at least output when the location of the coil lies outside the boundary region. It is based on the relative location of the coil and for example comprises a dataset about distance or twisting, a vector, which specifies how the coil must be moved towards the required position or simply information or a warning generated from the data.

An inventive apparatus for supporting an MRT examination of a patient via a coil placed on the patient, in particular for control of an MRT system, comprises the following components:
a data interface designed for receiving a protocol for the MRT examination and patient information comprising at least information about body dimensions and orientation and in particular also weight of the patient,
a measurement facility designed for measurement of a location of the coil via automated data acquisition,
a determination unit designed for automatic determination of an examination region of the patient from the protocol provided and the patient information, a required location of the coil above the examination region, and the location of the coil relative to the required location based on location of the coil determined,
a comparison unit designed for comparison of the relative location of the coil determined with a predetermined boundary region around predetermined required location of the coil,
a data interface designed for output of output data based on the relative location of the coil at least in the case that the location of the coil lies outside the boundary region.

The data interfaces can be grouped together into one bidirectional data interface or can be separate data interfaces.

The determination unit can comprise a number of determination modules for the different tasks or can be a single unit.

An inventive control facility for control of a magnetic resonance tomography system is designed for carrying out an inventive method and/or comprises an inventive apparatus.

An inventive magnetic resonance tomography system comprises an inventive control facility.

A large part of the components of the apparatus mentioned above or of the control facility can be realized entirely or partly in the form of software modules in a processor of a corresponding apparatus or control facility. A largely software-based realization has the advantage that even apparatuses or control facilities already used previously can be upgraded in a simple way by a software update in order to work in the inventive way. To this extent the object is also achieved by a corresponding computer program product with a computer program that is able to be loaded directly into a computing system or a memory facility of a control facility of a magnetic resonance tomography system, with program sections for carrying out all steps of one or more inventive methods when the program is executed in the computing system or the control facility. Such a computer program product, as well as the computer program, can where necessary comprise additional elements such as for example documentation and/or additional components including hardware components, such as for example hardware keys (dongles etc.) for use of the software.

A computer-readable medium, for example a memory stick, a hard disk or another transportable or permanently installed data medium can be used for transport to the computing system or to the control facility and/or for storage on or in the computing system or the control facility, on which computer-readable medium the program sections of the computer program able to be read in and executed by a computing system or a computer unit of the control facility are stored. The computer unit can for example have one or more interoperating microprocessors or the like for this purpose.

Further, one or more example embodiments of the present invention emerge for the dependent claims and also from the description given below, wherein the claims of one claim category can also be developed similarly to the claims and parts of the description for another claims category and in particular individual features of different exemplary embodiments or variants for new exemplary embodiments or variants can be combined.

In one preferred form of embodiment the coil is a flexible coil. Such coils are known in the prior art. “Flexible coil” means a deformable coil that can mold itself to the body contour of a patient or can be arranged around an area of the body. One or more example embodiments of the present invention is especially advantageous for flexible coils.

The location of a flexible coil can be well determined for example via analysis of images of a camera, attached above the patient table for example, in particular via software for object recognition (known in the prior art) or an arrangement of Hall effect sensors (for example in the corners or at the sides and if necessary in the center of the coil), which after corresponding one-time calibration, measures the known magnetic stray fields of the magnets of the magnetic resonance tomograph.

In one preferred form of embodiment the isocenter of the coil is determined from the measured location of the coil and a lateral distance of the isocenter from the examination region is preferably determined as the location of the coil. This can for example take place in a simple way by the position of a Hall effect sensor in the coil, in particular in the center of the coil, being determined or the isocenter being determined from camera images and an object recognition together with a measurement of the coil recognized in the image.

Preferably this distance is subsequently compared with a limit value. Output data is then preferably output when the distance is greater than the limit value. The limit value here would be the boundary region or part of the boundary region.

In one preferred form of embodiment a twisting of the coil in one plane or in space in relation to a predetermined coordinate system is determined from the measured location of the coil. This can be derived for example from the data of Hall effect sensors at different points of the coil or from camera images with an object recognition. This twisting is then compared with a predetermined maximum twisting in this coordinate system in the plane or in space as boundary region. Output data is preferably then output when the twisting of the coil is greater than the maximum twisting.

In one preferred form of embodiment the measurement facility comprises at least one measuring element of the group of Hall effect sensors, tilt sensors, cameras, in particular 3D cameras, ultrasound sensors, radar sensors and near field sensors. Preferably the measurement facility comprises one Hall effect sensor or an arrangement of Hall effect sensors, which are arranged on the coil, preferably at different points of the coil, for example on its corners and/or in the center and/or on the side edges.

A 3D camera could also serve to supply patient information. To this end the patient is determined in the camera data via automatic object recognition and their size or proportions and in particular also their weight are determined.

Furthermore a camera can additionally also be used to determine the routing of cables to the coil. The cable routing can certainly play a role in MRT measurement, in particular when the cable runs in a loop, which causes a very disadvantageous induction. In particular with PET-MRT measurements (PET: positron emission tomography) there is also the fact that the cable attenuates the photons and attention should also be paid to its guidance for this reason. A camera, in particular a 3D camera, can recognize the cable guidance and reproduce its route together with the location of the coil.

In accordance with one preferred form of embodiment of the invention the location of the coil and additionally the routing of the cable is taken into account in an image reconstruction and/or the output data is also based on the routing of the cable. In particular the output data comprises specifications for improved cable routing and/or a warning when the cable has an unsatisfactory course.

Moreover the cable guidance can also be taken into account in the reconstruction of images, in particular in the correction of the photon attenuation during a PET-MRT measurement.

In one preferred form of embodiment the output data is designed so that it comprises the location of the coil determined. What is meant by this is that the output data comprises specifications about the position and/or orientation of the coil relative to a predefined coordinate system. The output can also take place here when the boundary region has not been exceeded. Preferably, based on this output data, there is a reconstruction of raw data of the MRT examination. What this means is that for example a twisting of the coil and/or a shifting of the coil is taken into account during reconstruction of images. In particular a twisting of the coil should be taken into account in an accelerated reconstruction.

In one preferred form of embodiment there is a visualization of the coil based on the output data, preferably together with a visualization of the patient based on the patient information. What this preferably means is that the visualization takes place in the form of an avatar of the patient (also referred to as a body model or patient model) and the coil (or a virtual model of the coil). Preferably there is additionally a visualization of the examination region of the patient. Preferably in this case the display of the coil relative to the display of the patient or to their examination region corresponds to the relative location determined. This means that in the visualization the coil should have the same relative location in relation to the patient (and thus to the examination region) as it does in reality.

The location of the coil measured by a camera or a Hall effect sensor can be used for this purpose, in order to synchronize a virtual location of the coil with the location of the real coil with regard to the coordinate system, for example of the patient table. Then, with the patient data and a defined position of the patient, for example their head, it is possible to link the location of the virtual coil to a patient model, in order to obtain feedback of the actual spatial location of the coil at the expected location of the coil relative to the body part.

For a present, completed patient registration the body model can be employed as a database. Here it would be known to the system where the center of the coil must lie, so that an examination region of the patient, for example their right shoulder, is covered.

In one preferred form of embodiment, in the case that the location of the coil lies outside the boundary region or a measurement region of the MRT examination, a visual and/or acoustic warning is given. As an alternative or in addition information can also be output as to how the coil would have to be positioned so that the location of the coil lies within the boundary region and/or measurement region. A warning can for example be given on a screen and state “coil placed too far to the left”, “anatomy not sufficiently covered by the coil” or “twisting of the coil too great”. The output could also say “incorrect shoulder, position coil over right shoulder”. In a visualization of the location it could also be specified via arrows or an animation etc. where the coil should be positioned exactly on the avatar of a patient.

If the coil is positioned on a region that, because of a limited table movement path (with a part body scanner), cannot be moved into the isocenter or through restrictions of the magnetic field homogeneity should not be moved to, there is the opportunity with this warning already to warn the user at an earlier point in time that imaging in this region is not possible (“the region in which the coil lies cannot be moved into the isocenter!”). The aim is to inform the user at an early stage, so that an incorrect patient positioning can be avoided. This saves time for repositioning the patient.

After a manual shifting of the coil on the patient their location is subsequently preferably determined once again and the method is run once again. During this process a display of coil and patient is preferably continuously updated and at least adjusted to the current location of the coil. As soon as the expected location matches, there is then preferably a positive feedback to the user, for example “left shoulder OK”. Through this procedure it can also be established that the coil has accidentally been positioned on the wrong part of the body (for example right shoulder instead of left shoulder) and the user informed accordingly.

In one preferred form of embodiment the output data is used to adjust the protocol to the location of the coil, wherein the protocol is preferably adjusted so that the measurement is made in the isocenter of the coil. The examination in this case thus does not take place in the originally intended isocenter, but in that predetermined by the coil. This has the advantage the optimum measurement data is obtained thereby. It should be pointed out that a physician would like to measure an entirely specific region especially accurately when examining an organ and to do this the isocenter of the coil is deliberately placed exactly by the medical experts precisely over this region.

To do this it is preferred that the user has an opportunity to choose whether the measurement is to be made in the original isocenter or in the isocenter of the coil. Since the location of the coil is known at all times and the isocenter position is thus selected, the user could start the journey into the isocenter of the MRT scanner to do this. For this a particular selection switch could be pressed or a general selection switch could be pressed in a particular way (for example long press: 2 sec). However it does not absolutely have to start from the isocenter. The examination program used could also for example take account of the position of the coil edge that is closest to the scanner as the start, in particular when a larger region is covered by a number of flexible coils.

In one preferred form of embodiment, in the event that the location of the coil lies outside the boundary region, no MRT examination takes place. It is preferred in this regard that an MRT examination only takes place when the location of the coil lies within the boundary region or there has been a manual entry by an operator, which specifies that an MRT examination is still to be carried out (manual override). The type of the input in this case should preferably depend on the output data, since there can be incorrect coil positionings of different quality. If for example the coil location lies only slightly outside the boundary region, there could be a simple entry “still start measurement”. If on the other hand there is a dramatic mispositioning (for example left shoulder instead of right shoulder, coil outside the measurement region), then a particular input could be required, for example first an enabling with a password.

The use of an AI-based method (AI: Artificial Intelligence) for one or more inventive methods is preferred. An artificial intelligence is based on the principle of machine-based learning, and as a rule is carried out with an algorithm capable of learning that has been trained accordingly. The expression machine learning is frequently used for machine-based learning, wherein here also the principle of “deep learning” is also included in this. For example a deep convolutional neural network (DCNN) is trained to determine the coil position from camera images. The automatic recognition of objects in images is the prior art in this case.

The method has the advantage that it can recognize where the coil lies, for example by a Hall effect sensor in the coil or a 3D camera, and is beneficially integrated into the workflow of an MRT examination, in order to give users feedback about whether the position of the coil is correct, or if errors have been made, to rapidly make these transparent. The proposed method of operation in particular allows the manual selection of the isocenter position of the coil at the operator interface to be dispensed with, whereby the workflow becomes more efficient overall. The assistance resulting from the method confirms to (inexperienced) users that the coil positioning was correct and where necessary gives hints as to how the coil can be correctly positioned. This reduces mispositionings and saves time. Users are shown the problem clearly, they can learn and are thereby more efficient in their daily routine.

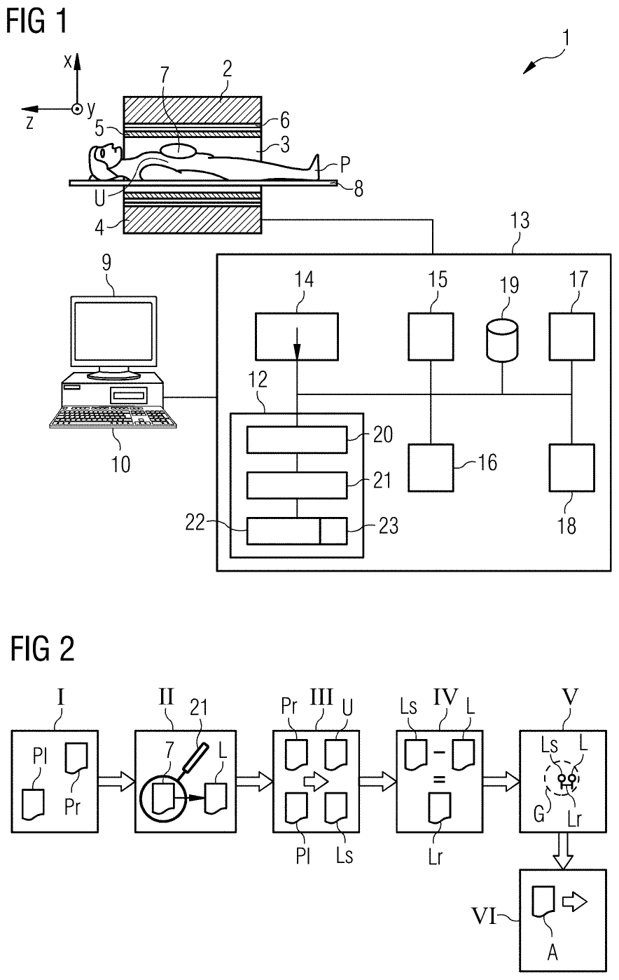

Shown as a rough schematic in FIG. 1 is a magnetic resonance tomography system 1. On the one hand it shows the actual magnetic resonance scanner 2 with an examination space 3 or patient tunnel, in which a patient P or test subject, in the body of which the examination region U is located, is positioned on a table 8.

The magnetic resonance scanner 2 is equipped in the usual way with a basic field magnet system 4, a gradient system 6 and also an RF transmit antenna system 5 and an RF receive antenna system 7. The exemplary embodiment shown the RF transmit antenna system 5 involves a whole-body coil permanently installed in the magnetic resonance scanner 2, whereas the RF receive antenna system 7 (in the form of a flexible coil 7) lies on the patient P here.

The basic field magnet system 4 is embodied here in the usual way so that it creates a basic magnetic field in the longitudinal direction of the patient P, i.e. along the longitudinal axis of the magnetic resonance scanner 2 running in the z direction. The gradient system 6 comprises individually activatable gradient coils in the usual way, in order to be able to switch gradients in the x, y or z direction independently of one another. Moreover the magnetic resonance scanner 2 contains shim coils (not shown), which can be embodied in the usual way.

The MRT system 1 shown here involves a whole-body installation with a patient tunnel, into which a patient P can be introduced entirely. However the invention can also basically be used on other MRT systems, for example with a C-shaped housing open to the side. What is important is only that the corresponding images of the examination region U can be produced.

The magnetic resonance tomography system 1 furthermore has a central control facility 13 that is used for control of the MRT system 1. This central control facility 13 comprises a sequence control unit 14. With said unit the sequence of radio-frequency pulses (RF pulses) and of gradient pulses is controlled as a function of a chosen pulse sequence or of a series of a number of pulse sequences for imaging a number of slices in a volume region of interest of the examination object within a measurement session. Such a pulse sequence can be predetermined of parameterized within a measurement or control protocol (abbreviated to: “protocol” Pr). Usually different protocols Pr are stored for different measurements and measurement sessions in a memory 19 and can be selected by a user (and if required modified where necessary) and only then used for carrying out the measurement. In the present case the control facility 13 contains pulse sequences for acquisition of the raw data.

To output the individual RF pulses of a pulse sequence the central control facility 13 has a radio-frequency transmit facility 15, which creates the RF pulses, amplifies them and feeds them via a suitable interface (not shown in detail) into the RF transmit antenna system 5. For control of the gradient coils of the gradient system 6, in order, according to the predetermined pulse sequence, to switch the gradient pulses in an appropriate way, the control facility 13 has a gradient system interface 16. Via this gradient system interface 16 the diffusion gradient pulses and spoiler gradient pulses could be applied. The sequence control unit 14 communicates in a suitable way with the radio-frequency transmit facility 15 and the gradient system interface 16 to carry out the pulse sequence.

The control facility 13 moreover has a radio-frequency receive facility 17 (likewise communicating in a suitable way with the sequence control unit 14), in order, within the readout window predetermined by the pulse sequence, coordinated via the RF receive antenna system 7, to acquire magnetic resonance signals and in this way the raw data.

A reconstruction unit 18 takes over the acquired raw data here and reconstructs magnetic resonance image data from it. This reconstruction too takes place as a rule on the basis of parameters, which can be predetermined in the respective measurement and control protocol. This image data can then for example be stored in a memory 19.

The details of how, by radiating in RF pulses and switching gradient pulse, suitable raw data can be acquired and MR images or parameter maps can be reconstructed from this, are basically known to the person skilled in the art and will therefore not be explained in any greater detail here.

The control facility 13 furthermore has an apparatus 12 for supporting an MRT examination of a patient P via the coil 7 placed on the patient P. This apparatus 12 comprises the following components:

A data interface 20, which is designed for receiving a protocol Pr for the MRT examination and patient information PI comprising at least one item of information about body dimensions and orientation (on the table 8) of the patient P.

A measurement facility 21, for example an arrangement of Hall effect sensors 21 or a camera, designed for measurement of a location of the coil 7 via automated data acquisition.

A determination unit 22, for example in the form of a computing unit, designed for automatic determination of the examination region U of the patient P from the protocol Pr provided and the patient information PI, a required location of the coil 7 above the examination region U, and the relative location of the coil 7 in relation to the required location.

A comparison unit 23 (here part of the determination unit 22) designed to compare the relative location of the coil 7 determined with a predetermined boundary region around a predetermined required location of the coil 7.

The data interface 20 here is likewise designed for output of output data A based on the relative location of the coil 7.

The central control facility 13 can be operated via a terminal 11 with an input unit 10 and a display unit 9, via which the entire magnetic resonance tomography system 1 can thus also be operated by an operator. Magnetic resonance tomography images and visualizations can also be displayed on the display unit 9 within the framework of the invention, and measurements can be planned and started via the input unit 10, if necessary in combination with the display unit 9 and in particular control protocols can be selected and where necessary modified.

What is more, the inventive magnetic resonance tomography system 1 and in particular the control facility 13 can have a plurality of further components, not shown here in detail, but usually present in these types of installation, such as for example a network interface, in order to connect the system as a whole to a network and to be able to exchange raw data and/or image data or parameter maps, but also further data, such as for example patient-relevant data or control protocols.

The way in which, by radiating in RF pulses and creating gradient pulses, suitable raw data can be acquired and magnetic resonance tomography images can be reconstructed from this, is basically known to the person skilled in the art and will therefore not be explained in any greater detail here.

As stated, the MRT system shown here is merely one possible example. A further preferred example would be a PET MRT system, which represents a combination of magnetic resonance tomography (MRT) and Positron Emission Tomography (PET). There too receive coils not located in one fixed position can be used.

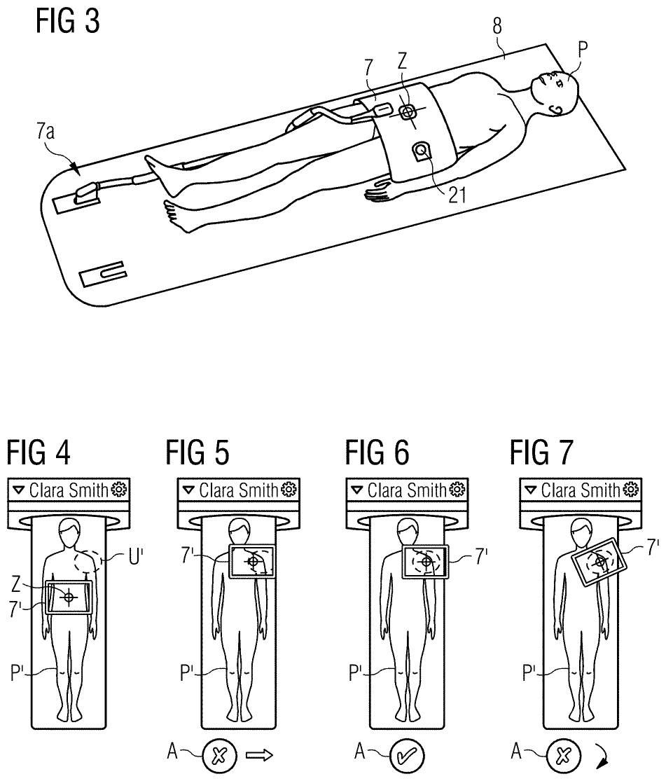

FIG. 2 shows a flow diagram for a possible execution sequence of an inventive method for supporting an MRT examination of a patient P via a coil 7 placed on the patient P (see for example FIG. 1 or 3).

In step I a protocol Pr for the MRT examination and patient information PI comprising at least information about body dimensions and orientation on the table 8 and in particular also the weight of the patient P is provided.

In step II the location L of the coil 7 is measured via automated data acquisition by a measurement facility 21.

In step III there is an automatic determination of an examination region U of the patient P from the protocol Pr provided and the patient information PI and of a required location Ls of the coil 7 above the examination region U of the patient.

In step IV there is an automatic determination of the location Lr of the coil 7 relative to the required location Ls based on the location L of the coil determined.

In step V the relative location Lr of the coil 7 determined is compared with a predetermined boundary region G around a predetermined required location Ls of the coil 7.

In step VI there is an output of output data A based on the relative location Lr of the coil 7 at least in the case that the location L of the coil 7 lies outside the boundary region G.

FIG. 3 shows a patient P with a flexible coil 7 on a couch 8. The coil is linked via connection 7a (a cable 7a) to the scanner 2 (see FIG. 1), so that the signals measured with it can be included in the imaging. The coil has a Hall effect sensor 21 as its measurement facility 21. However said coil does not lie in the isocenter Z of the coil 7, but its position relative to the isocenter Z (and to other positions of the coil 7) is known.

In particular when a camera is used in the (or as the) measurement facility 21, the routing of the cable 7a can be determined here and taken into account by a warning in one inventive method.

FIG. 4 shows an example for a display of a patient P′, a coil 7′, and an examination region U′. Located in the center of the coil is the isocenter Z. The real coil 7 here still lies outside the examination region U on the patient P, which is expressed accordingly in the diagram.

For reasons of improved clarity the isocenter Z and the representation of the examination region U′ will no longer be labeled below with reference characters. The examination region U will always be the left shoulder below, the isocenter Z always in the center of the coil 7.

FIG. 5 shows an example for an incorrectly positioned coil 7. The representation of the coil 7′ lies, corresponding to the real coil, only at the edge of the examination region U (or of the examination region U′ shown). Below the representation a warning is output ('X′) as output data A together with an indication (arrow) of the point to which the coil 7 would have to be shifted. Despite this, through a manual override, a measurement could still be carried out, for example when precisely this area of the examination region is to be mapped especially well.

FIG. 6 shows an example for a correctly positioned coil 7. The representation of the coil 7′ now lies, corresponding to the real coil, over the examination region U (or the examination region U′ displayed). According to FIG. 5 an operator could have positioned the coil 7 correctly. Below the representation a notification (tick) is output as output data A, to show that the positioning is now correct. To do this for example the method has been carried out again and the location of the shifted coil 7 checked and displayed.

FIG. 7 shows an example for a twisted coil 7. The representation of the coil 7′ again lies, corresponding to the real coil, above the examination region U (or the examination region U′ shown). According to FIG. 5 an operator could now have positioned the coil 7 incorrectly instead of as shown in FIG. 6. Below the representation a warning is output ('X′) as output data A together with a notification (arrow) of the point to which the coil 7 would have to be twisted.

Although some example embodiments of the present invention have been disclosed in the form of preferred embodiments and variations thereon, it will be understood that numerous additional modifications and variations could be made thereto without departing from the scope of example embodiments of the present invention. For the sake of clarity, it is to be understood that the use of “a” or “an” throughout this application does not exclude a plurality, and “comprising” does not exclude other steps or elements. The mention of a “unit”, “module” or a “device” does not preclude the use of more than one unit or device.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’, ‘unit’, interface' or the term ‘controller’ may be replaced with the term ‘circuit.’ The term ‘module’ and may ‘unit’ refer to, be part of, or include processor hardware (shared, dedicated, or group) that executes code and memory hardware (shared, dedicated, or group) that stores code executed by the processor hardware.

The module or interface may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices (i.e., storage means). The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

The invention is not limited to the example embodiments described hereintofore. Rather, other variants of the invention can also be derived herefrom by the person skilled in the art, without departing from the subject matter of the invention. In particular, it is furthermore possible to combine all the individual features described in connection with the example embodiment with one another in a different way also, without departing from the subject matter of the invention.

In conclusion it is pointed out once again that the method and also the magnetic resonance tomography system 1 shown in detail merely involve exemplary embodiments, which can be modified in a wide variety of ways by the person skilled in the art, without departing from the area of the invention. Furthermore the use of the indefinite article “a” or “an” does not exclude the features concerned also being able to be present a number of times. Likewise the terms “unit” and “apparatus” do not exclude the components concerned consisting of a number of interoperating sub-components, which if necessary can also be spatially distributed.

1. A method for supporting an MRT examination of a patient via a coil placed on the patient, comprising:
providing of a protocol for the MRT examination;
providing patient information, the patient information comprising at least information about body dimensions and orientation of the patient;
measuring a location of the coil via an automated data acquisition by a measurement facility;
automatically determining an examination region of the patient from the protocol and the patient information provided and a required location of the coil;
automatically determining the location of the coil relative to the required location based on the location of the coil measured;
comparing the relative location of the coil determined with a predetermined boundary region around a predetermined required location of the coil; and
outputting output data based on the relative location of the coil when the relative location of the coil lies outside the boundary region.

2. The method as claimed in claim 1, wherein the coil is a flexible coil.

3. The method of claim 1, wherein
the measuring the location of the coil includes,
determining an isocenter of the coil from the measured location of the coil, a lateral distance from the isocenter to the examination region being the location of the coil, and the outputting includes,
outputting the lateral distance when the lateral distance is greater than a limit value.

4. The method of claim 1, further comprising:
determining a twisting of the coil in one plane or in space with regard to a predetermined coordinate system based on the measured location of the coil; and
comparing the determined twisting with a predetermined maximum twisting in the predetermined coordinate system, wherein the outputting outputs the output data when the determining twisting is greater than the maximum twisting.

5. The method of claim 1, wherein the measurement facility comprises at least one measurement element, the measurement element being a Hall effect sensor, a Tilt sensor, a camera, an ultrasound sensor, a radar sensor or a near field sensor, the at least one measurement element is arranged on at least one of the coil or a camera.

6. The method of claim 1, wherein the output data includes the location of the coil determined.

7. The method of claim 1, further comprising:
providing a visualization of the coil based on the output data.

8. The method of claim 1, wherein the outputting outputs at least one of a visual warning, an acoustic warning or information when the relative location of the coil lies outside the boundary region or a measurement region of the MRT examination, at least one of a visual warning, an acoustic warning or information indicating how the coil is to be positioned such that the relative location of the coil lies within at least one of the boundary region or a measurement region.

9. The method of claim 1, wherein the output data is used to adjust the protocol to the location of the coil.

10. The method of claim 1, wherein an MRT examination only takes place when the relative location of the coil lies within the boundary region or there is a manual input by an operator.

11. An apparatus for supporting an MRT examination of a patient via a coil placed on the patient, comprising:
a first data interface configured to receive a protocol for the MRT examination and patient information, the patient information comprising at least information about body dimensions and orientation of the patient;
a measurement facility configured to measure a location of the coil via automated data acquisition;
a determination unit configured to automatically determine an examination region of the patient from the protocol determined and the patient information, a required location of the coil above the examination region, and the location of the coil relative to the required location based on the location L of the coil determined;
a comparison unit configured to compare the relative location of the coil determined with a predetermined boundary region around a predetermined required location of the coil; and
a second data interface configured to output of output data based on the relative location of the coil when the location of the coil lies outside the boundary region.

12. A control facility for controlling a magnetic resonance tomography system, which is configured to carry out the method of claim 1.

13. A magnetic resonance tomography system comprising the control facility of claim 12.

14. A non-transitory computer program product with a computer program with program sections for carrying out the method of claim 1 when the computer program is executed in a computing system or a control facility of a magnetic resonance tomography system.

15. A non-transitory computer-readable medium including program sections that, when executed by a computer unit, cause the computer unit perform the method of claim 1.

16. The method of claim 2, further comprising:
determining a twisting of the coil in one plane or in space with regard to a predetermined coordinate system based on the measured location of the coil; and
comparing the determined twisting with a predetermined maximum twisting in the predetermined coordinate system, wherein the outputting outputs the output data when the determining twisting is greater than the maximum twisting.

17. The method of claim 3, further comprising:
determining a twisting of the coil in one plane or in space with regard to a predetermined coordinate system based on the measured location of the coil; and
comparing the determined twisting with a predetermined maximum twisting in the predetermined coordinate system, wherein the outputting outputs the output data when the determining twisting is greater than the maximum twisting.

18. The method of claim 2, further comprising:
providing a visualization of the coil based on the output data.

19. The method of claim 16, further comprising:
providing a visualization of the coil based on the output data.

20. The method of claim 17, further comprising:
providing a visualization of the coil based on the output data.
